(19) United States
(12) Patent Application Publication
(10) Pub. No.: US 2023/0200420 A1
(43) Pub. Date: Jun. 29, 2023

(54) EDIBLE FUNGI

(71) Applicant: MARLOW FOODS LIMITED, Stokesley (GB)

(72) Inventors: Olumuyiwa A. AKINTOYE, Stokesley Yorkshire (GB); Alexander James EVANS, Stokesley Yorkshire (GB); Jonathan Paul MILLER, Stokesley Yorkshire (GB)

(21) Appl. No.: 17/926,929

(22) PCT Filed: May 14, 2021
(86) PCT No.: PCT/GB2021/051163

§ 371 Date: Nov. 21, 2022

(30) Foreign Application Priority Data
May 22, 2020 (GB) 2007676.6

(51) Int. Cl. A23L 31/00; C12N 1/14; A23L 5/20

(52) U.S. Cl. CPC A23L 31/00; C12N 1/14; A23L 5/23; C12R 2001/77

(57) ABSTRACT

An edible mass comprising fungal particles of a filamentous fungus which can more readily be used in production of sweet products and/or has reduced mushroom taste whilst maintaining desired nutritional attributes is described. The mass may include relatively low levels of acetyl-glutamic acid, uridine, inosine, guanosine, UMP, GMP, AMP, sodium, potassium and/or ammonium.

This invention relates to edible fungi and particularly, although not exclusively, relates to an edible mass comprising fungal particles of a filamentous fungus which may be used as a food ingredient in a range of foodstuffs.

It is known, for example from WO00/15045 (DSM), WO96/21362 (Zeneca) and WO95/23843 (Zeneca) to use edible filamentous fungi as meal-substitutes, for example in the preparation of burgers and sausages. The fact that the edible filamentous fungi have a mushroom taste may be of little consequence in some savoury foodstuffs such as the aforesaid. In any event, the mushroom taste may be masked by use of a range of flavorants. However, the mushroom taste may make it difficult to produce consumer-acceptable sweet or non-savoury products such as milks, ice-cream or other deserts incorporating filamentous fungi. Even if consumer-acceptable sweets or non-savoury products can be produced, this will often only be achievable by use of relatively high amounts of flavorants and/or other taste-masking ingredients. It may also be desirable, even in some savoury products, to reduce levels of flavorants used in general and/or which may be used to mask the mushroom taste.

It is an object of preferred embodiments of the present invention to address the aforementioned problems.

It is an object of preferred embodiments of the present invention to provide an edible mass comprising fungal particles of a filamentous fungus which has reduced mushroom taste whilst maintaining desired nutritional attributes.

It is an object of preferred embodiments of the present invention to reduce the amount of ingredients needed to mask a mushroom taste in foodstuffs comprising a filamentous fungus

It is an object of preferred embodiments of the present invention to provide an edible mass comprising fungal particles of a filamentous fungus which can more readily be used in production of sweet products.

Unless otherwise specified “parts per million” or “ppm” refers to the number of units of mass of a specified component per million units of a total specified mass. Thus, ppm is suitably quoted on a weight for weight basis.

According to a first aspect of the invention, there is provided an edible mass comprising fungal particles of a filamentous fungus, wherein said mass includes at least one of the following characteristics (a) to (j):
(a) less than 0.7 ppm of acetyl-glutamic acid on a wet matter basis and/or less than 2.9 ppm of acetyl-glutamic acid on a dry matter basis;
(b) less than 40 ppm uridine on a wet matter basis and/or less than 160 ppm of uridine on a dry matter basis;
(c) less than 18 ppm inosine on a wet matter basis and/or less than 75 ppm of inosine on a dry matter basis;
(d) less than 150 ppm guanosine on a wet matter basis and/or less than 625 ppm of guanosine on a dry matter basis;
(e) less than 160 ppm UMP on a wet matter basis and/or less than 660 ppm of UMP on a dry matter basis;
(f) less than 200 ppm GMP on a wet matter basis and/or less than 800 ppm of GMP on a dry matter basis;
(g) less than 210 ppm AMP on a wet matter basis and/or less than 875 ppm of AMP on a dry matter basis;
(h) less than 80 ppm sodium on a wet matter basis and/or less than 330 ppm of sodium on a dry matter basis;
(i) less than 560 ppm potassium on a wet matter basis and/or less than 2300 ppm of potassium on a dry matter basis;
(j) less than 370 ppm ammonium on a wet matter basis and/or less than 1500 ppm of ammonium on a dry matter basis.

The reference to characteristics on a wet matter basis suitably means that the amount of water included in the mass is taken into consideration in calculating the ppm. The reference to characteristics on a dry matter basis suitably means that the amount of water included in the mass is ignored in calculating the ppm.

Characteristics and/or components described herein may be assessed as described in the examples.

In a first preferred embodiment, said mass includes at least one of the following characteristics (a) to (j):
(a) less than 0.4 ppm of acetyl-glutamic acid on a wet matter basis and/or less than 1.6 ppm of acetyl-glutamic acid on a dry matter basis;
(b) less than 20 ppm uridine on a wet matter basis and/or less than 80 ppm of uridine on a dry matter basis;
(c) less than 12 ppm inosine on a wet matter basis and/or less than 50 ppm of inosine on a dry matter basis;
(d) less than 100 ppm guanosine on a wet matter basis and/or less than 400 ppm of guanosine on a dry matter basis;
(e) less than 100 ppm UMP on a wet matter basis and/or less than 400 ppm of UMP on a dry matter basis;
(f) less than 100 ppm GMP on a wet matter basis and/or less than 400 ppm of GMP on a dry matter basis;
(g) less than 100 ppm AMP on a wet matter basis and/or less than 400 ppm of AMP on a dry matter basis;
(h) less than 80 ppm sodium on a wet matter basis and/or less than 330 ppm of sodium on a dry matter basis;
(i) less than 250 ppm potassium on a wet matter basis and/or less than 1000 ppm of potassium on a dry matter basis;
(j) less than 250 ppm ammonium on a wet matter basis and/or less than 1000 ppm of ammonium on a dry matter basis.

In a second preferred embodiment, said mass includes at least one of the following characteristics (a) to (j):
(a) less than 0.2 ppm of acetyl-glutamic acid on a wet matter basis and/or less than 0.8 ppm of acetyl-glutamic acid on a dry matter basis;
(b) less than 10 ppm uridine on a wet matter basis and/or less than 42 ppm of uridine on a dry matter basis;
(c) less than 8 ppm inosine on a wet matter basis and/or less than 40 ppm of inosine on a dry matter basis;
(d) less than 30 ppm guanosine on a wet matter basis and/or less than 120 ppm of guanosine on a dry matter basis;
(e) less than 40 ppm UMP on a wet matter basis and/or less than 160 ppm of UMP on a dry matter basis;
(f) less than 50 ppm GMP on a wet matter basis and/or less than 200 ppm of GMP on a dry matter basis;
(g) less than 50 ppm AMP on a wet matter basis and/or less than 200 ppm of AMP on a dry matter basis;
(h) less than 80 ppm sodium on a wet matter basis and/or less than 300 ppm of sodium on a dry matter basis;
(i) less than 250 ppm potassium on a wet matter basis and/or less than 1000 ppm of potassium on a dry matter basis;
(j) less than 250 ppm ammonium on a wet matter basis and/or less than 1000 ppm of ammonium on a dry matter basis.

In a third preferred embodiment, said mass includes at least one of the characteristics (a) to (g).

Said mass may include at least two of the characteristics (a) to (j). Said mass may include at least six of the characteristics (a) to (j). Said mass may include all of the characteristics (a) to (j).

Said mass may include at least two of the characteristics (a) to (g). Said mass may include at least four of the characteristics (a) to (g). Said mass may include all of the characteristics (a) to (g).

Said mass may include at least one, preferably each, of the characteristics (e) to (g).

For each characteristic referred to, said mass preferably includes the specified characteristic on both a wet and dry matter basis. In a preferred embodiment, said mass includes the specified characteristic on a dry matter basis.

Said mass preferably includes at least one of the following characteristics, wherein an amount of a component is specified per 100 g of said edible mass on a wet matter basis:
(A) at least 1 g/100 g of aspartic acid;
(B) at least 0.5 g/100 g of serine;
(C) at least 1.5 g/100 g of glutamic acid;
(D) at least 0.5 g/100 g of glycine;
(E) at least 0.25 g/100 g of histidine;
(F) at least 0.7 g/100 g of arginine;
(G) at least 0.5 g/100 g of threonine;
(H) at least 0.6 g/100 g of alanine;
(I) at least 0.4 g/100 g of proline;
(J) at least 0.07 g/100 g of cystine;
(K) at least 0.3 g/100 g of tyrosine;
(L) at least 0.7 g/100 g of valine;
(M) at least 0.2 g/100 g of methionine;
(N) at least 1.0 g/100 g of lysine;
(O) at least 0.5 g/100 g of iso-leucine;
(P) at least 0.8 g/100 g of leucine
(Q) at least 0.4 g/100 g of phenylalanine;
(R) at least 0.15 g/100 g of tryptophan.

Said mass may include at least five, preferably at least ten, more preferably at least fifteen of characteristics (A) to (R).

Said mass preferably includes at least one of the following characteristics, wherein an amount of a component is specified per 100 g of said edible mass on a dry matter basis:
(S) at least 4.1 g/100 g of aspartic acid;
(T) at least 2.0 g/100 g of serine;
(U) at least 6.0 g/100 g of glutamic acid;
(V) at least 2.0 g/100 g of glycine;
(VV) at least 1.0 g/100 g of histidine;
(X) at least 2.8 g/100 g of arginine;
(Y) at least 2.0 g/100 g of threonine;
(Z) at least 0.6 g/100 g of alanine;
(AA) at least 2.4 g/100 g of proline;
(BB) at least 0.2 g/100 g of cystine;
(CC) at least 1.2 g/100 g of tyrosine;
(DD) at least 2.8 g/100 g of valine;
(EE) at least 0.8 g/100 g of methionine;
(FF) at least 4.0 g/100 g of lysine;
(GG) at least 2.0 g/100 g of iso-leucine;
(HH) at least 3.2 g/100 g of leucine
(II) at least 1.6 g/100 g of phenylalanine;
(JJ) at least 0.6 g/100 g of tryptophan.

Said mass may include at least five, preferably at least ten, more preferably at least fifteen of characteristics (S) to (JJ).

Said mass preferably comprises particles of said filamentous fungus (herein also referred to as “fungal particles”). Said filamentous fungus preferably comprises fungal mycelia and suitably at least 80 wt %, preferably at least 90 wt %, more preferably at least 95 wt % and, especially, at least 99 wt % of the fungal particles in said mass comprise fungal mycelia. Some filamentous fungi may include both fungal mycelia and fruiting bodies. Said fungal particles preferably comprise a filamentous fungus of a type which does not produce fruiting bodies. Where, however, a filamentous fungus of a type which produces fruiting bodies is used, the fungal particles in said mass suitably include at least 80 wt %, preferably at least 90 wt %, more preferably at least 95 wt % of fungal mycelia. Preferably, said fungal particles comprise substantially only fungal mycelia—that is, said fungal particles in said mass preferably do not include any fruiting bodies.

Preferred fungi for said fungal particles have a cell wall which includes chitin and/or chitosan. Preferred fungi have a cell wall which includes polymeric glucosamine. Preferred fungi have a cell wall which includes β1-3 and 1-6 glucans.

Said fungal particles preferably comprise (preferably consist essentially of) fungus, for example selected from fungi imperfecti.

Preferably, said fungal particles comprise, and preferably consist essentially of, cells of Fusarium species, especially of Fusarium venenatum A3/5 (formerly classified as Fusarium graminearum) (IMI 145425; ATCC PTA-2684 deposited with the American Type Culture Collection, 10801 University Boulevard, Manassas, Va.) as described for example in WO96/21361 (Zeneca) and WO95/23843 (Zeneca).

Preferably, said fungal particles are non-viable. Preferably, said fungal particles have been treated to lower the level of RNA which they contain. Thus, the level of RNA in the fungal particles used is preferably less than the level in an identical fungus when in a viable state.

The level of RNA in the fungal particles is preferably less than 2 wt % on a dry matter basis.

Fungal particles in said mass may comprise filaments having lengths of less than 1000 μm, preferably less than 800 μm. Said filaments may have a length greater than 100 μm, preferably greater than 200 μm. Preferably, fewer than 5 wt %, preferably substantially no, fungal particles in said mass have lengths of greater than 5000 μm; and preferably fewer than 5 wt %, preferably substantially no, fungal particles have lengths of greater than 2500 μm. Preferably, values for the number average of the lengths of said fungal particles in said mass are also as stated above.

Fungal particles in said mass may comprise filaments having diameters of less than 20 μm, preferably less than 10 μm, more preferably 5 μm or less. Said filaments may have diameters greater than 1 μm, preferably greater than 2 μm. Preferably, values for the number average of said diameters of said fungal particles in said mass are also as stated above.

Fungal particles in said mass may comprise filaments having an aspect ratio (length/diameter) of less than 1000, preferably less than 750, more preferably less than 500, especially of 250 or less. The aspect ratio may be greater than 10, preferably greater than 40, more preferably greater than 70. Preferably, values for the average aspect ratio of said fungal particles (i.e. the average of the lengths of the particles divided by the average of the diameters of the fungal particles) in said mass are also as stated above.

Said mass may comprise said filamentous fungus and water which is suitably homogenous. The mass is preferably in the form of a paste (suitably a homogenous paste) which is suitably flowable. The viscosity of said paste at 800 Pa and 10° C. may be at least 5000 Pa/s, preferably at least 8000 Pa/s. The viscosity of said paste at 800 Pa and 10° C. may be less than 20000 Pa/s, preferably less than 13000 Pa/s. Said mass may comprise at least 10 wt % and, preferably, less than 40 wt %, of said filamentous fungus on a dry matter basis. Said mass may comprise at least 60 wt % and, preferably, less than 90 wt % of water. The ratio defined as wt % of water in said mass divided by the wt % of filamentous fungus in said mass (on a dry matter basis) may be in the range 2 to 4. Said mass may comprise 10 to 40 wt % (preferably 20 to 30 wt %) of filamentous fungus on a dry matter basis and 60 to 90 wt % (preferably 70 to 80 wt %) of water.

The sum of the wt % of said filamentous fungus and water in said mass is suitably at least 90 wt %, preferably at least 95 wt %, more preferably at least 99 wt %.

According to a second aspect of the invention, there is provided a foodstuff for human consumption, said foodstuff comprising an edible mass according to the first aspect mixed with one or more other ingredients.

In a first preferred embodiment, said edible mass includes fungal particles, suitably as herein described, which include at least one of the following characteristics:
(k) less than 1.6 ppm of acetyl-glutamic acid on a dry matter basis;
(l) less than 80 ppm of uridine on a dry matter basis;
(m) less than 50 ppm of inosine on a dry matter basis;
(n) less than 400 ppm of guanosine on a dry matter basis;
(o) less than 400 ppm of UMP on a dry matter basis;
(p) less than 400 ppm of GMP on a dry matter basis;
(q) less than 400 ppm of AMP on a dry matter basis;
(r) less than 330 ppm of sodium on a dry matter basis;
(s) less than 1000 ppm of potassium on a dry matter basis;
(t) less than 1000 ppm of ammonium on a dry matter basis.

In a second preferred embodiment, said edible mass includes fungal particles, suitably as herein described, which include at least one of the following characteristics:
(k) less than 0.8 ppm of acetyl-glutamic acid on a dry matter basis;
(l) less than 42 ppm of uridine on a dry matter basis;
(m) less than 40 ppm of inosine on a dry matter basis;
(n) less than 120 ppm of guanosine on a dry matter basis;
(o) less than 160 ppm of UMP on a dry matter basis;
(p) less than 200 ppm of GMP on a dry matter basis;
(q) less than 200 ppm of AMP on a dry matter basis;
(r) less than 300 ppm of sodium on a dry matter basis;
(s) less than 1000 ppm of potassium on a dry matter basis;
(t) less than 1000 ppm of ammonium on a dry matter basis.

Said edible mass preferably includes fungal particles, suitably as herein described, which include at least one of the following characteristics, wherein an amount of a component is specified per 100 g of said fungal particles on a dry matter basis:
(KK) at least 4.1 g/100 g of aspartic acid;
(LL) at least 2.0 g/100 g of serine;
(MM) at least 6.0 g/100 g of glutamic acid;
(NN) at least 2.0 g/100 g of glycine;
(OO) at least 1.0 g/100 g of histidine;
(PP) at least 2.8 g/100 g of arginine;
(QQ) at least 2.0 g/100 g of threonine;
(RR) at least 0.6 g/100 g of alanine;
(SS) at least 2.4 g/100 g of proline;
(TT) at least 0.2 g/100 g of cystine;
(UU) at least 1.2 g/100 g of tyrosine;
(VV) at least 2.8 g/100 g of valine;
(WW) at least 0.8 g/100 g of methionine;
(XX) at least 4.0 g/100 g of lysine;
(YY) at least 2.0 g/100 g of iso-leucine;
(ZZ) at least 3.2 g/100 g of leucine
(AAA) at least 1.6 g/100 g of phenylalanine;
(BBB) at least 0.6 g/100 g of tryptophan.

Said fungal particles may include at least five, preferably at least ten, more preferably at least fifteen of characteristics (KK) to (BBB).

Said edible mass may be mixed with an ingredient (A).

Ingredient (A) may be selected from:
(i) a puree (e.g., bean puree, sweet potato puree, pumpkin puree, applesauce, yam puree, banana puree, plantain puree, date puree, prune puree, fig puree, zucchini puree, carrot puree, coconut puree);
(ii) native or modified starches (e.g., starches from grains, starches from tuber, potato starch, sweet potato starch, corn starch, waxy corn starch, tapioca starch, tapioca, arrowroot starch, taro starch, pea starch, chickpea starch, rice starch, waxy rice starch, lentil starch, barley starch, sorghum starch, wheat starch, and physical or chemical modifications thereof [including, e.g., pre-gelatinized starch, acetylated starch, phosphate bonded starch, carboxymethylated starch, hydroxypropylated starch]);
(iii) flours derived from grains or legumes or roots (e.g., from taro, banana, jackfruit, konjac, lentil, fava, lupin bean, pea, bean, rice, wheat, barley, rye, corn, sweet rice, soy, teff, buckwheat, amaranth, chickpea, sorghum, almond, chia seed, flaxseed, potato, tapioca, potato);
(iv) protein isolates (e.g., from potato, soy, pea, lentil, chickpea, lupin, oat, canola, wheat), hydrolyzed protein isolates (e.g., hydrolyzed pea protein isolate, hydrolyzed soy protein isolate);
(v) protein concentrates (e.g. from algae, lentil, pea, soy, chickpea, rice, hemp, fava bean, pigeon pea, cowpea, vital wheat gluten);
(vi) gums (e.g., xanthan gum, guar gum, locust bean gum, gellan gum, gum arabic, vegetable gum, tara gum, tragacanth gum, konjac gum, fenugreek gum, gum karaya, gellan gum, high-acetyl gellan gum, low-acetyl gellan gum);
(vii) native or relatively folded (i.e., not fully in the native functional state but not fully denatured) proteins (e.g., fava protein, lentil protein, pea protein, ribulose-1,5-bisphosphate carboxylase/oxygenase [Rubisco], chickpea protein, mung bean protein, pigeon pea protein, lupin bean protein, soybean protein, white bean protein, black bean protein, navy bean protein, adzuki bean protein, sunflower seed protein);
(viii) polysaccharides and modified polysaccharides (e.g., methylcellulose, hydroxypropyl methylcellulose, carboxymethyl cellulose, maltodextrin, carrageenan and its salts, alginic acid and its salts, agar, agarose, agaropectin, pectin, alginate).

Said ingredient (A) may be derived from a non-animal source. Said ingredient (A) may be derived from a plant.

Said foodstuff may incorporate other ingredients, for example one or more flavouring materials.

Said foodstuff may be part of a food product for human consumption, for example selected from mince, a burger, a sausage, or meat-like pieces or strips. In one preferred embodiment, said foodstuff may be non-savoury. It may be a drink, such as a milk or sweet solid foodstuff.

According to a third aspect of the invention, there is provided a method of making an edible mass according to the first aspect and/or as described in the second aspect, the method comprising:
(i) selecting a precursor mass comprising fungal particles of a filamentous fungus;
(ii) contacting said precursor mass with an aqueous solvent to produce a mixture;
(iii) filtering said mixture;
(iv) isolating a residue which comprises said edible mass.

It is found that the aqueous solvent extracts selected components from the precursor mass, including, advantageously, components responsible for the savoury and/or mushroom taste of the filamentous fungus. In addition, advantageously, the level of amino acids/protein in said filamentous fungus is not significantly reduced by the process which means that the process importantly does not significantly diminish the nutritional value of the edible mass produced.

In step (ii), preferably, said precursor mass and aqueous solvent are agitated, suitably to intimately mix the precursor mass and solvent and facilitate a reduction in the level of undesirable components remaining in the edible mass. Preferably, agitation does not involve high shear but is preferably arranged not to significantly affect the dimensions of the fungal particles. Thus, a ratio of the average lengths of fungal particles isolated in step (iv) divided by the average lengths of fungal particles in said precursor mass selected in step (i) is at least 0.7, preferably at least 0.9. Said ratio may be about 1.

Suitably, after step (ii), the aqueous solvent entrains components extracted from the fungal particles. Upon filtration in step (iii), the filtrate suitably contains said aqueous solvent and components extracted from the fungal particles. Consequently, the residue which suitably defines the edible mass has a reduced level of certain components (which have been extracted into the aqueous solvent as described). It is found that the components extracted include many of those responsible for the mushroom-taste of the fungal particles.

Consequently, the edible mass produced advantageously has a reduced mushroom taste/smell.

Said aqueous solvent selected in step (ii), preferably includes at least 70 wt %, preferably at least 95 wt %, more preferably at least 99 wt % water. Said aqueous solvent selected in step (ii), preferably consists essentially of water. It may consist of substantially pure water. It may consist of distilled or deionised water.

A filtrate produced after filtration of said mixture may include one or more components selected from:
I acetyl-glutamic acid;
II uridine;
III inosine;
IV guanosine;
V UMP;
VI GMP;
VII AMP;
VIII sodium;
IX potassium;
X ammonium.

Said filtrate produced after filtration of said mixture may include at least three, at least seven or all of the components selected from I to X above.

Said filtrate produced after filtration of said mixture may include one or more of components II to VII. Said filtrate produced after filtration of said mixture may include at least three, at least five, or all of components II to VII.

A ratio defined as the total weight of protein contained in the residue divided by the total weight of protein contained in the filtrate is at least 1, is preferably at least 2 and may be at least 5 or at least 10. Thus, the majority of the protein is not extracted into the aqueous solvent and advantageously remains associated with the edible mass. Proteins may be assessed, for example, a spectroscopic Biuret test.

Said precursor mass comprising fungal particles of a filamentous fungus selected in step (i), may be as described in the first aspect.

According to a fourth aspect of the invention, there is provided a method of making a foodstuff of the second aspect, the method comprising:
(a) selecting a mass of the first aspect and/or a mass made in a method of the third aspect; and
(b) contacting said mass with one or more other ingredients.

According to a fifth aspect, there is provided the use of a mass of the first aspect and/or a mass made in a method of the third aspect in making a foodstuff with a reduced mushroom taste and/or flavour.

Any feature of any aspect of any invention described herein may be combined with any feature of any other invention described herein mutatis mutandis.

Specific embodiments of the invention will now be described, by way of example, with reference to the accompanying figures, in which:

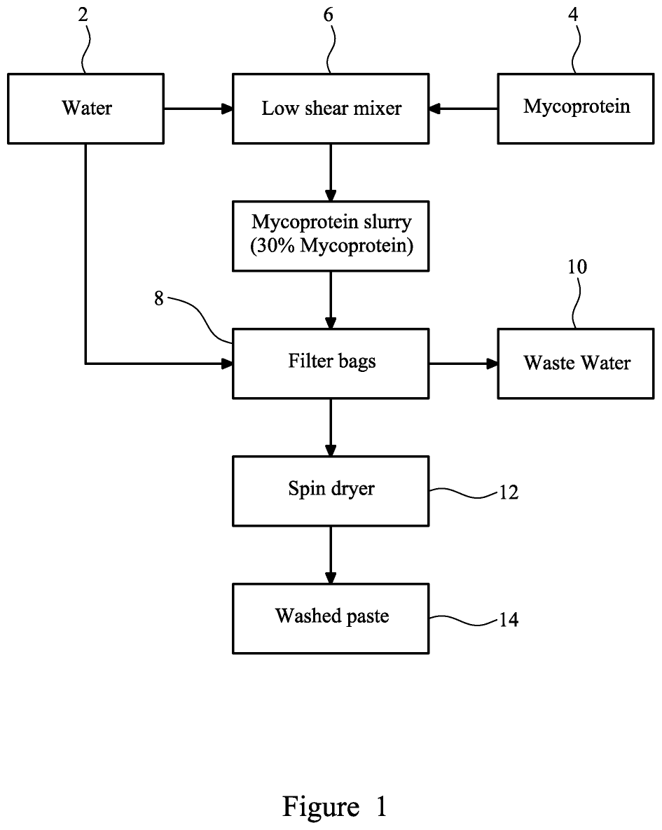

FIG. 1 is a schematic diagram of a first process for washing mycoprotein paste;

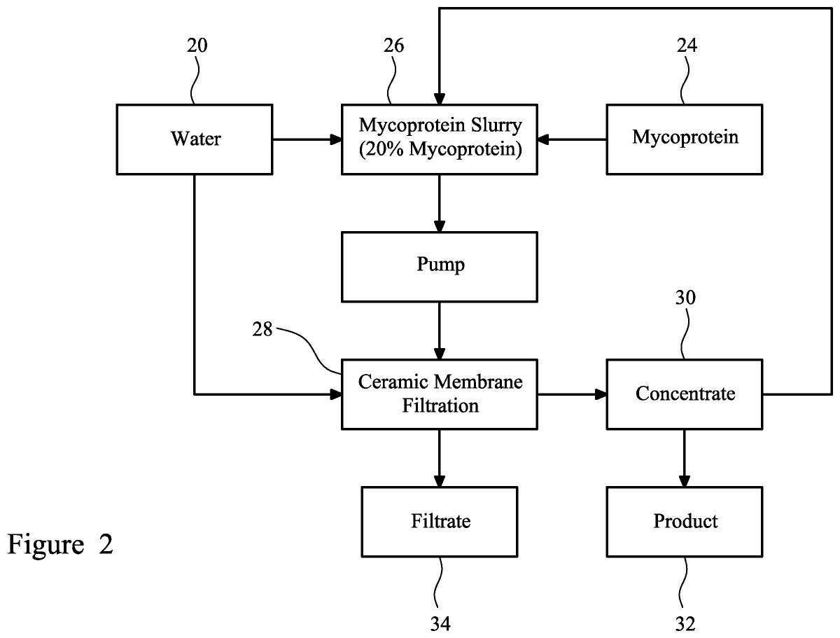

FIG. 2 is a schematic diagram of a second process for washing mycoprotein paste; and

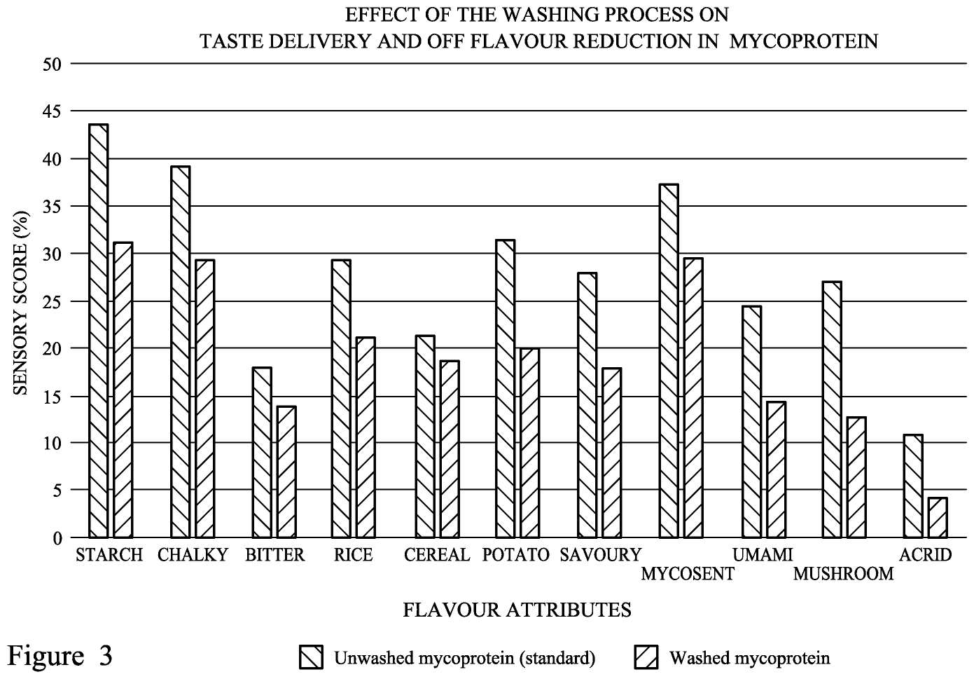

FIG. 3 is a diagram which illustrates the effect of washing on taste as assessed by a trained panel.

The following material is referred to hereinafter:

Mycoprotein paste—Mycoprotein paste-refers to a visco-elastic material comprising a mass of edible filamentous fungus derived from Fusarium venenatum A3/5 (formerly classified as Fusarium graminearum Schwabe) (IMI 145425; ATCC PTA-2684 deposited with the American type Culture Collection, 12301 Parklawn Drive, Rockville Md. 20852) and treated to reduce its RNA content to less than 2% by weight by heat treatment. Further details on the material are provided in WO96/21362 and WO95/23843. The material may be obtained from Marlow Foods Limited of Stokesley, U.K. It comprises about 23-25 wt % solids (the balance being water) made up of non-viable RNA reduced fungal hyphae of approximately 400-750 μm length, 3-5 μm in diameter and a branching frequency of 2-3 tips per hyphal length.

UMP, GMP and AMP refer to uridine monophosphate, guanosine monophosphate and adenosine monophosphate respectively.

The following tests methods are used to analyse mycoprotein paste.

### Test 1—Analysis of Paste Residual Molecules Before and After Washing

### Material Preparation

A sample of mycoprotein was mixed with water to a ratio of 1 part mycoprotein to 10 parts water. De-ionised water was used for extraction. The mix of sample and water was introduced to a homogenizer (Polytron GT 10-35) and homogenized at high shear at 10-15k rotations for 1 min. This action produced a slurry. The slurry was filtered through two syringe-filters in a row. The first filter was a Spartan™ 30/0.45RC and the second was a Millex GN Nylon 0.2 μm. The supernatant was then then analysed.

### Analysis

1. Anions and organic acids (Lactate, Acetate, Formate, Chloride, Sulphate, malate, Succinate, Phosphate, Citrate)
   Instrument: High-Performance Ion Chromatography (HPIC). The ICS-3000 ion chromatography system (Dionex, Olten, Switzerland) consisted of two ICS-300 DP pumps (isocratic and gradient), an ICS-3000 autosampler, a DC ICS-3000 thermal compartment, and an amperometric and an electrochemical detector. System control and data acquisition were performed using Chromeleon software (version 6.7, Dionex).
   Anions were analyzed using an lonPac AS11-HC analytical column (4×250 mm, Dionex) equipped with an lonPac AG11-HC guard column (4×50 mm, Dionex) and self-regenerating anion suppressor ASRS-Ultra II (4 mm, Dionex) operating at 223 mA with hydroxide eluent generation. Chromatography was performed at 30° C. with a flow rate of 1.5 mL/min using aqueous potassium hydroxide as solvent and starting with a concentration of 1 mM for 1 min, increasing the ion strength to 30 mM within 14 min, then to 60 mM within 10 min, and maintaining this concentration for 7 min.
2. Cations (Sodium, Ammonium, Potassium, Magnesium, Calcium)
   Instrument: see Anions.
   Cations were analyzed using an lonPac CS12-A analytical column (4×250 mm, Dionex) equipped with an lonPac CG12-A guard column (4×50 mm, Dionex) and self-regenerating cation suppressor CSRS-Ultra II (4 mm, Dionex) operating at 88 mA with methanesulfonic acid eluent generation. Isocratic chromatography was performed at 30° C. with a flow rate of 1.5 mL/min using aqueous methanesulfonic acid with a concentration of 20 mM for 12 min.
3. Nucleotides (Adenine, Uridine, Adenosine, Guanosine)
   Instrument: High-Performance Liquid Chromatography (HPLC). For analytical HPLC, an Agilent 1100 series HPLC system consisting of a binary pump, an autosampler, a column oven (at 30° C.), an online degasser, and a diode array detector (Agilent, Waldbronn, Germany) was used. Data acquisition was performed using the software HP ChemStation (Agilent, Waldbronn, Germany). Nucleotides were analyzed using an RP-HPLC-DAD method. Therefore, a defined amount of the Quorn was dissolved in deionized water and membrane filtered (0.45 μm). Aliquots were injected onto an RP18 column (Zorbax Eclipse, 150×4.6, 5 μm, Agilent, Santa Clara, Calif., USA) and separated with a gradient of methanol/acetonitrile (5:4; v/v; solvent A) and 23 mM (NH4)2HPO4 in water (pH 6.0, solvent B). Using a flow rate of 1.0 mL/min, chromatography was started with 100% B; in 25 min the content of A was increased to 30% and maintained for 15 min at that solvent ratio. Detection was performed by means of a DAD set at 254 nm. For quantification six-point external calibration curves were recorded.
4. Other constituents (Homoserine, Pyrogluatmic acid, Strombine, Glutamic acid)
   Instrument: High-Performance Liquid Chromatography—Mass Spectrometry (HPLC-MS/MS). The Agilent 1200 series HPLC system, consisting of a binary pump, an online degasser, a column oven (at 30° C.), and an autosampler (Agilent), was connected to an API 3200 QTRAP mass spectrometer (AB Sciex Instruments, Darmstadt, Germany), which was equipped with an electrospray ionization (ESI) source and operated in the positive ionization mode. The ion spray voltage was set to 3500 or 4000 V depending on the HPLC method (HILIC, 3500 V; PFP, 4000 V), and the declustering potential and the MS/MS parameters were optimized for each substance to induce fragmentation of the pseudo molecular ion [M−H]+ to the corresponding target product ions after collision-induced dissociation. The dwell time for each mass transition was 150 ms, and the declustering potential (DP), the cell exit potential (CXP), and the collision energy (CE) were optimized for each substance. Quantitative analysis was performed by means of the multiple reaction monitoring (MRM) mode using the fragmentation parameters optimized prior to analysis. Data processing and integration were performed by using Analyst software version 1.5.1 (AB Sciex Instruments). For the MRM-IDA-EPI MS experiments an information-dependent acquisition (IDA) method using these MRM survey scans to confirm the presence and identity of the NAG was applied. In the case of the presence of the target analyte, a full scan enhanced product ion (EPI) spectrum of the compound was acquired, and this mass spectrum was compared to the one of the reference compounds. To quantitate some additional taste compounds, two LC-MS/MS multimethods based on two orthogonal HPLC columns (PFP-RP18 and ZIC-HILIC) were developed. A defined amount of the Quorn extract was dissolved in deionized water and membrane filtered (0.20 μm). Aliquots were analyzed on a SeQuant ZIC-HILIC column (150×4.6 mm, 5 μm, SeQuant, Ume& Sweden) and on a Phenomenex Luna PFP column (250×4.6 mm, 3 μm, Phenomenex, Aschaffenburg, Germany) via MS/MS. In both cases the target compounds were analyzed by means of HPLC-MS/MS operating in the MRM with positive electrospray ionization.

### Test 2—Analysis of Protein Content

This was undertaken using a standard method.

### Test 3—Analysis of Fat Content

This was undertaken using a standard method.

The following examples describe how edible fungal particles with reduced levels of specified components may be prepared.

### Example 1—Washing of Mycoprotein Paste—First Method (Lab Method)

Referring to FIG. 1, water 2 and mycoprotein paste 4 are contacted and mixed in a low shear mixer 6 to produce a slurry which includes 30 wt % mycoprotein paste. After mixing, the slurry is filtered using filter bags 8 to produce waste water filtrate 10 and solids which are spin dried 12 to produce washed paste 14.

### Example 2—Washing of Mycoprotein Paste—Second Method (Ceramic Membrane Method)

Referring to FIG. 2, water 20 and mycoprotein paste 24 are contacted and mixed and a slurry 26 produced comprising 20 wt % mycoprotein. The slurry is pumped through a ceramic filtration membrane with pore size 0.1 micron at pressures between 4-6 bar, in the presence of additional water. A concentrate 30 is produced and the product 32 comprising washed paste isolated. Waste filtrate 34 is also collected.

### Example 3—Washing of Mycoprotein Paste—Third Method (Vacuum Belt)

The process of Example 2 is followed except that a vacuum filter belt (7 micron) is used to filter the mycoprotein slurry instead of the ceramic filtration membrane of Example 2.

The washed paste of Examples 1, 2 and/or 3 were analysed as described in Tests 1, 2 and/or 3 and results are provided below.

### Results

Table 1 details results of analyses, following the procedures referred to in Tests 1 to 3 of mycoprotein which has been washed using the processes of Examples 1 to 3. The table also details the amounts of specified compounds/molecules in mycoprotein prior to any washing. The results are quoted based on the amount of mycoprotein on a wet matter basis.

TABLE 1

| | | Mycoprotein | Mycoprotein | Mycoprotein |
|---|---|---|---|---|
| | Unwashed | washed as per | washed as per | washed as per |
| | Mycoprotein | Example 1 | Example 2 | Example 3 |
| Compound/Molecule | (mg/kg paste) | (mg/kg paste) | (mg/kg paste) | (mg/kg paste) |
| UMP | 165.00 | 17.10 | 0.00 | 12.50 |
| GMP | 214.00 | 27.30 | 11.00 | 45.20 |
| AMP | 227.00 | 24.80 | 0.00 | 0.00 |
| Sodium | 81.00 | 76.00 | 77.00 | 112.00 |
| Ammonium | 381.00 | 0.00 | 0.00 | 42.00 |
| Potassium | 564.00 | 129.00 | 166.00 | 264.00 |
| Magnesium | 139.00 | 43.00 | 47.00 | 73.00 |
| Calcium | 107.00 | 40.00 | 60.00 | 66.00 |
| Homoserine | 5.15 | 2.40 | 5.27 | 1.51 |
| Nicotineamide | 0.48 | 0.68 | 0.00 | 0.00 |
| Pyroglutamic acid | 4.93 | 1.87 | 0.58 | 2.17 |
| Hypoxanthine | 16.28 | 0.73 | 4.70 | 25.11 |
| Uridine | 42.30 | 4.94 | 12.10 | 19.75 |
| Adenosine | 37.08 | 8.68 | 7.57 | 0.56 |
| Inosine | 18.69 | 5.48 | 7.40 | 8.07 |
| Guanosine | 158.76 | 16.22 | 40.20 | 25.95 |
| Acetyglutamic acid | 0.79 | 0.09 | 0.20 | 0.39 |

As a result of Applicant's assessment, it is believed certain compounds/molecules contribute most significantly to the mushroom flavour of the mycoprotein which it is desired to reduce. Table 2 details such compounds/molecules contained in unwashed mycoprotein and in mycoprotein washed as described in Example 1. The results are quoted based on the amount of mycoprotein on a wet matter basis.

TABLE 2

| | Unwashed | Mycoprotein |
|---|---|---|
| | Mycoprotein | washed as per |
| Compound/Molecule/Ion | (mg/kg) | Example 1 |
| UMP | 165.00 | 17.10 |
| GMP | 214.00 | 27.30 |
| AMP | 227.00 | 24.80 |
| Ammonium ions | 381.00 | 0.00 |
| Potassium ions | 564.00 | 129.00 |
| Uridine | 42.30 | 4.94 |
| Inosine | 18.69 | 5.48 |
| Guanosine | 158.76 | 16.22 |
| Acetylglutamic acid | 0.79 | 0.09 |

Mycoprotein is used as a foodstuff and, more particularly, as a source of dietary protein. Consequently, it is desirable that any treatment does not reduce the amount of amino acids/protein within the mycoprotein after the treatments described. Table 3 details results of analysis of the levels of certain amino acids in unwashed mycoprotein and in mycoprotein washed as described in Example 1. The results are quoted based on the amount of mycoprotein on a wet matter basis.

TABLE 3

| | | Mycoprotein |
|---|---|---|
| | Unwashed | washed as per |
| Analysis | Mycoprotein | Example 1 |
| Aspartic Acid (g/100 g) | 1.36 | 1.47 |
| Serine (g/100 g) | 0.65 | 0.70 |
| Glutamic Acid | 1.67 | 1.72 |
| (g/100 g) | | |
| Glycine (g/100 g) | 0.65 | 0.69 |
| Histidine (g/100 g) | 0.33 | 0.36 |
| Arginine (g/100 g) | 0.93 | 1.01 |
| Threonine (g/100 g) | 0.71 | 0.77 |
| Alanine (g/100 g) | 0.87 | 0.90 |
| Proline (g/100 g) | 0.60 | 0.64 |
| Cystine (g/100 g) | 0.09 | 0.10 |
| Tyrosine (g/100 g) | 0.38 | 0.41 |
| Valine (g/100 g) | 0.83 | 0.92 |
| Methionine (g/100 g) | 0.25 | 0.27 |
| Lysine (g/100 g) | 1.12 | 1.21 |
| Iso-Leucine (g/100 g) | 0.67 | 0.73 |
| Leucine (g/100 g) | 0.99 | 1.07 |
| Phenylalanine | 0.51 | 0.56 |
| (g/100 g) | | |
| Tryptophan (g/100 g) | 0.20 | 0.22 |

It has been found that the processes described do not significantly detrimentally affect the nutritional value of the mycoprotein—the mycoprotein is found, after washing as described, to be calorifically almost identical to the unwashed mycoprotein and to include levels of fat, carbohydrate and protein comparable with the unwashed mycoprotein.

Mycoprotein treated as described in Example 1 was assessed for taste delivery and off-flavour reduction by a trained panel of individuals who assessed a range of flavour attributes. Results are provided in FIG. 3, wherein the unwashed (standard mycoprotein) is shown on the left and the washed mycoprotein is shown on the right for each attribute. It should be noted that, for each of the attributes assessed, the savoury score is reduced, meaning that the particular flavour attribute is reduced in the washed protein.

Advantageously, mycoprotein which has been washed as described has a less savoury and/or mushroom flavour. Consequently, it may be used in foodstuffs where it is desired to minimise such flavours However, other important nutritional characteristics, such as amino acids and proteins are not detrimentally reduced.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. An edible mass comprising fungal particles of a filamentous fungus, wherein said mass includes at least one of the following characteristics (a) to (j):
(a) less than 0.7 ppm of acetyl-glutamic acid on a wet matter basis and/or less than 2.9 ppm of acetyl-glutamic acid on a dry matter basis;
(b) less than 40 ppm uridine on a wet matter basis and/or less than 160 ppm of uridine on a dry matter basis;
(c) less than 18 ppm inosine on a wet matter basis and/or less than 75 ppm of inosine on a dry matter basis;
(d) less than 150 ppm guanosine on a wet matter basis and/or less than 625 ppm of guanosine on a dry matter basis;
(e) less than 160 ppm UMP on a wet matter basis and/or less than 660 ppm of UMP on a dry matter basis;
(f) less than 200 ppm GMP on a wet matter basis and/or less than 800 ppm of GMP on a dry matter basis;
(g) less than 210 ppm AMP on a wet matter basis and/or less than 875 ppm of AMP on a dry matter basis;
(h) less than 80 ppm sodium on a wet matter basis and/or less than 330 ppm of sodium on a dry matter basis;
(i) less than 560 ppm potassium on a wet matter basis and/or less than 2300 ppm of potassium on a dry matter basis;
(j) less than 370 ppm ammonium on a wet matter basis and/or less than 1500 ppm of ammonium on a dry matter basis.

2. (canceled)

3. A mass according to claim 1, wherein said mass includes at least four of the following characteristics (a) to (g):
(a) less than 0.8 ppm of acetyl-glutamic acid on a dry matter basis;
(b) less than 42 ppm of uridine on a dry matter basis;
(c) less than 40 ppm of inosine on a dry matter basis;
(d) less than 120 ppm of guanosine on a dry matter basis;
(e) less than 160 ppm of UMP on a dry matter basis;
(f) less than 200 ppm of GMP on a dry matter basis;
(g) less than 200 ppm of AMP on a dry matter basis.

4. (canceled)

5. A mass according to claim 1, wherein said mass includes each of the characteristics (e) to (g).

6. A mass according to claim 1, wherein said mass includes at least ten of the following characteristics, wherein an amount of a component is specified per 100 g of said edible mass on a dry matter basis:
(A) at least 4.1 g/100 g of aspartic acid;
(B) at least 2.0 g/100 g of serine;
(C) at least 6.0 g/100 g of glutamic acid;
(D) at least 2.0 g/100 g of glycine;
(E) at least 1.0 g/100 g of histidine;
(F) at least 2.8 g/100 g of arginine;
(G) at least 2.0 g/100 g of threonine;
(H) at least 0.6 g/100 g of alanine;
(I) at least 2.4 g/100 g of proline;
(J) at least 0.2 g/100 g of cystine;
(K) at least 1.2 g/100 g of tyrosine;
(L) at least 2.8 g/100 g of valine;
(M) at least 0.8 g/100 g of methionine;
(N) at least 4.0 g/100 g of lysine;
(O) at least 2.0 g/100 g of iso-leucine;
(P) at least 3.2 g/100 g of leucine
(Q) at least 1.6 g/100 g of phenylalanine;
(R) at least 0.6 g/100 g of tryptophan.

7. (canceled)

8. A mass according to claim 1, wherein said mass comprises particles of said filamentous fungus and said fungal particles comprise cells of Fusarium species.

9. (canceled)

10. A mass according to claim 1, wherein the level of RNA in the fungal particles is less than 2 wt % on a dry matter basis.

11. A mass according to claim 1, wherein said mass comprises at least 40 wt % of said filamentous fungus on a dry matter basis.

12. A mass according to claim 1, wherein the sum of the wt % of said filamentous fungus and water in said mass is at least 99 wt %.

13. A foodstuff for human consumption, said foodstuff comprising an edible mass according to claim 1, mixed with one or more other ingredients.

14. A foodstuff according to claim 13, which includes at least one of the following characteristics:
(k) less than 1.6 ppm of acetyl-glutamic acid on a dry matter basis;
(l) less than 80 ppm of uridine on a dry matter basis;
(m) less than 50 ppm of inosine on a dry matter basis;
(n) less than 400 ppm of guanosine on a dry matter basis;
(o) less than 400 ppm of UMP on a dry matter basis;
(p) less than 400 ppm of GMP on a dry matter basis;
(q) less than 400 ppm of AMP on a dry matter basis;
(r) less than 330 ppm of sodium on a dry matter basis;
(s) less than 1000 ppm of potassium on a dry matter basis;
(t) less than 1000 ppm of ammonium on a dry matter basis; and
wherein said edible mass is an ingredient (A), which is selected from: a puree; native or modified starches; flours; protein isolates; protein concentrates; gums; native or relatively folded proteins; polysaccharides and modified polysaccharides.

15. (canceled)

16. (canceled)

17. A foodstuff according to claim 13, wherein said foodstuff is non-savoury.

18. A foodstuff according to claim 13, wherein said foodstuff is a milk or a sweet solid foodstuff.

19. A method of making an edible mass according to claim 1, the method comprising:
(i) selecting a precursor mass comprising fungal particles of a filamentous fungus;
(ii) contacting said precursor mass with an aqueous solvent to produce a mixture;
(iii) filtering said mixture;
(iv) isolating a residue which comprises said edible mass.

20. A method according to claim 19, wherein said aqueous solvent selected in step (ii) includes at least 99 wt % water.

21. A method according to claim 19, wherein a filtrate produced after filtration of said mixture includes one or more components selected from:
I. acetyl-glutamic acid;
II. uridine;
III. inosine;
IV. guanosine;
V. UMP;
VI. GMP;
VII. AMP;
VIII. sodium;
IX. potassium;
X. ammonium.

22. A method according to claim 21, wherein said filtrate produced after filtration of said mixture includes all of the components I to X; and/or wherein a ratio defined as the total weight of protein contained in the residue divided by the total weight of protein contained in the filtrate is at least 10.

23. (canceled)

24. (canceled)

25. (canceled)

26. A mass according to claim 1, wherein said mass includes each of the following characteristics (e) to (g):
(e) less than 160 ppm of UMP on a dry matter basis;
(f) less than 200 ppm of GMP on a dry matter basis;
(g) less than 200 ppm of AMP on a dry matter basis; and
wherein said mass comprises fungal particles which comprise cells of Fusarium species.

27. A mass according to claim 1, wherein the level of RNA in the fungal particles in less than 2 wt % on a dry matter basis, wherein said mass comprises at least 40 wt % of said filamentous fungus on a dry matter basis, and wherein the sum of the wt % of said filamentous fungus and water in said mass is at least 99 wt %.

28. A mass according to claim 1, wherein said fungal particles comprise cells of Fusarium venenatum A3/5.

29. A mass according to claim 26, wherein the level of RNA in the fungal particles is less than 2 wt % on a dry matter basis, wherein said mass comprises at least 40 wt % of said filamentous fungus on a dry matter basis, wherein the sum of the wt % of said filamentous fungus and water in said mass is at least 99 wt %, and wherein said fungal particles comprise cells of Fusarium venenatum A3/5.
